(19) United States
(12) Patent Application Publication
(10) Pub. No.: US 2019/0106726 A1
(43) Pub. Date: Apr. 11, 2019

(54) BIOLOGICAL INDICATOR

(71) Applicant: AMERICAN STERILIZER COMPANY, Mentor, OH (US)

(72) Inventors: Tricia Cregger, Fairlawn, OH (US); William Yirava, Willoughby, OH (US); Phillip Franciskovich, Concord, OH (US); Kathleen A. Fix, Willoughby, OH (US)

(21) Appl. No.: 15/729,691

(22) Filed: Oct. 11, 2017

(51) Int. Cl. C12Q 1/22; A61L 2/20

(52) U.S. Cl. CPC C12Q 1/22; A61L 2/208

(57) ABSTRACT

This invention relates to a biological indicator, comprising: a carrier; and a spore deposit on the carrier, wherein at least about 95% of the area of the spore deposit comprises spores residing in a single spore layer.

### TECHNICAL FIELD

This invention relates to a biological indicator for monitoring the effectiveness of a sterilization process.

### BACKGROUND

Biological indicators, which typically comprise a carrier and test microorganisms (e.g., bacterial spores) deposited on the carrier, are used to monitor the effectiveness of sterilization processes. The biological indicator is placed in a sterilization chamber and subjected to a sterilization cycle along with the load intended for sterilization (e.g., a medical device). Following the sterilization cycle, the biological indicator is exposed to a growth media and incubated for the purpose of determining if any of the test microorganisms are viable. A successful sterilization cycle is indicated by a complete inactivation (no outgrowth) of the test microorganisms. An unsuccessful sterilization cycle is indicated by an incomplete inactivation (outgrowth detected) of the test microorganisms.

### SUMMARY

A problem in the art relates to the fact that biological indicators are typically designed to be sterile prior to the end of a sterilization cycle. That is, they are designed in such a manner that for a successful sterilization all of the spores on the biological indicator will be killed prior to the end of the sterilization cycle. In some cases, the biological indicators are designed not to exceed the half cycle (i.e., half way through a typical exposure phase during a sterilization cycle). However, many biological indicators become sterile much earlier than at the half cycle due in part to significant spore kill in pre-exposure conditioning phases. For this reason, there are often significant portions of the exposure phase during a sterilization cycle that are not monitored by live spores. This can lead to problems in determining whether a sterilization is successful.

One solution to this problem involves increasing the population bioload of the spores on the carrier. This can be achieved by depositing an inoculum containing a high concentration of the spores on the carrier. This results in a significant delay until all the spores are killed. A problem with this approach, however, relates to the fact that this often leads to uncontrolled spore stacking. Spore stacking occurs when spores overlie one another such that spores on top protect spores underneath from exposure to a sterilant during a sterilization cycle. Too much spore stacking can lead to false positives where some of the underlying spores survive the full cycle of a sterilization process even though conditions for a successful sterilization are met.

This invention provides a solution to this problem. With this invention it is possible to combine sufficient resistance to extend coverage further into the exposure phase of a sterilization cycle and simultaneously ensure that the spores do not survive the end of a successful sterilization cycle due to excess stacking. This invention involves increasing the population of the spores on the carrier in a way in which spore stacking is significantly reduced or eliminated.

This invention relates to a biological indicator, comprising: a carrier; and a spore deposit on the carrier, wherein at least about 95%, or at least about 96%, or at least about 97%, or at least about 98%, or at least about 99%, or at least about 99.5%, or at least about 99.8%, or at least about 99.9%, or at least about 99.95%, or at least about 99.99%, of the area of the spore deposit comprises spores residing in a single spore layer. In an embodiment, less than about 5%, or less than about 4%, or less than about 3%, or less than about 2%, or less than about 1%, or less than about 0.5%, or less than about 0.1%, or less than about 0.05%, or less than about 0.01% of the area of the spore deposit comprises spores residing in a stacked configuration. The spore deposit may have a total number of spores in the range from about 1×10⁶ to about 1×10⁸ colony-forming units (cfu), or from about 1×10⁷ to about 1×10⁸ cfu.

In an embodiment, this invention relates to a biological indicator, comprising: a carrier; and a spore deposit on the carrier, the spore being derived from an inoculum comprising the spores, water, and optionally a surfactant. The spore deposit may be formed by depositing the inoculum on the carrier and then drying the inoculum leaving the spore deposit on the carrier. In an embodiment, the inoculum forms a perimeter upon being deposited on the carrier, and prior to being dried the inoculum may be spread to expand the perimeter. In an embodiment, the inoculum includes a surfactant which allows for uniform lay down of the spore desposit over a wider area as compared to when no surfactant is used. In either case, it is possible to form the inventive biological indicator with little or no spore stacking as described above.

### BRIEF DESCRIPTION OF THE DRAWINGS

FIG. 1 is a perspective view of a self-contained biological indicator (SCBI) which can be used in accordance with the present invention.

FIG. 2 is a cross-sectional view of the SCBI of FIG. 1 (taken along the line 2-2 in FIG. 1) showing a cap mounted on a container in a first non-activated position;

FIG. 3 is a cross-sectional view of the SCBI of FIG. 1 (taken along the line 2-2 in FIG. 1) showing the cap mounted on the container in a second activated position.

FIG. 4 is a cross-sectional view of the SCBI of FIG. 1 (taken along line 4-4 in FIG. 1) showing the indicator in a second activated position.

FIG. 5 is a side-top perspective view of a test pack that may be used with an SCBI in accordance with the invention.

FIG. 6 is a cross-sectional view of the test pack illustrated in FIG. 5 taken along lines 6-6 of FIG. 5.

FIG. 7 is a top plan view of a test pack that can be used in accordance with the present invention, with an SCBI in a first recessed compartment and a chemical integrator and/or chemical indicator in a second recessed compartment.

FIG. 8 is a schematic illustration of spore deposits from (A) a 20 microliter drop of an inoculum containing spores dispersed in water, and (B) a 20 microliter drop of an inoculum containing spores and a surfactant dispersed in water. These spore deposits are described in Example 1.

FIG. 9 is a low magnification photomicrograph of the spore deposit from the (A) a 20 microliter drop of spores dispersed in water illustrated in FIG. 8 and described in Example 1.

FIG. 10 is a 40× magnification photomicrograph of the spore deposit shown in FIG. 9.

FIG. 11 is a 200× magnification photomicrograph of the spore deposit shown in FIG. 9.

FIG. 12 is a low magnification photomicrograph of the spore deposit from (B) a 20 microliter drop (B) containing the spores and a surfactant dispersed in water illustrated in FIG. 8 and described in Example 1.

FIG. 13 is a 40× magnification photomicrograph of the spore deposit shown in FIG. 12.

FIG. 14 is a 200× magnification photomicrograph of the spore deposit shown in FIG. 12.

FIG. 15 is a low magnification photomicrograph of a spore deposit from a microliter droplet (C) containing spores dispersed in water, the deposit having been spread as described in Example 2.

FIG. 16 is a 40× magnification photomicrograph of the spore deposit shown in FIG. 15.

### DETAILED DESCRIPTION

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

The term “spore stacking” refers to a spore deposit wherein spores overlie one another such that spores on top protect spores underneath from exposure to a sterilant during a sterilization process. Spore stacking can be detected using light and electron microscopy.

The term “single spore layer” refers to a layer of spores wherein no spore stacking occurs. A spore deposit with a single spore layer may have a thickness of one spore. A single spore layer may be referred to as a monodispersed spore layer. With this invention spore deposits are used wherein at least about 95%, or at least about 96%, or at least about 97%, or at least about 98%, or at least about 99%, or at least about 99.5%, or at least about 99.8%, or at least about 99.9%, or at least about 99.95%, or at least about 99.99%, of the area of the spore deposit comprises spores residing in a single spore layer. In an embodiment, less than about 5%, or less than about 4%, or less than about 3%, or less than about 2%, or less than about 1%, or less than about 0.5%, or less than about 0.1%, or less than about 0.05%, or less than about 0.01% of the area of the spore deposit comprises spores residing in a stacked configuration.

The term “area of the spore deposit” refers to the area of a spore deposit that is measured after an inoculum containing the spores is deposited on the carrier and dried. Drying refers to sufficient drying to allow the spore deposit to be used as a biological indicator in a sterilization process. An area of a spore deposit can be determined during production on an ink jet printer by monitoring the deposition using a fiduciary camera and knowing the concentration of the inoculum dispensed by the printer (in the nano to picoliter range) or by use of a light microscope to get an overall view of the deposit or by an electron microscope which allows resolution of individual spores.

The term “biological indicator” refers to a microbiological test device which comprises a carrier and a test microorganism in the form of spores deposited on the carrier.

The term “carrier” refers to a support onto which spores can be deposited to form a biological indicator.

The term “inoculated carrier” refers to a carrier onto which spores have been deposited.

The term “inoculum” refers to a composition containing spores dispersed in water which can be used to inoculate a carrier. The inoculum may further comprise a surfactant. The inoculum may be used to form a biological indicator by depositing the inoculum on a carrier, and then drying the inoculum leaving a spore deposit on the carrier.

The term “killing” spores refers to rendering spores incapable of reproduction, metabolism and/or growth.

The spores used with the inventive biological indicator may be used as test microorganisms during a sterilization cycle wherein the spores are more resistant to the sterilant than the organisms to be killed by the sterilization process. The killing of the spores during a sterilization cycle is indicative of a successful sterilization cycle.

The term “log reduction” is a mathematical term to show the number of live spores killed by contacting the spores with a sterilant during a sterilization cycle. A “4 log reduction” means that the number of live spores at the end of the sterilization process is 10,000 times smaller than the number of live spores prior to commencement of the sterilization process. A “5 log reduction” means that the number of live spores is 100,000 times smaller. A “6 log reduction” means that the number of live spores is 1,000,000 times smaller.

The term “D-value” or “decimal reduction value” refers to the time required to achieve inactivation of 90% of a population of spores (also known as a 1 log reduction). The D-value may be expressed in terms of seconds. With this invention, the D-value for the inventive biological indicator may be in the range from about 10 to about 50 seconds, or about 15 to about 40 seconds, or about 20 to about 35 seconds, or about 25 to about 35 seconds.

The term “sterilization” may refer to a total absence of living spores that remain after the completion of a sterilization process or sterilization cycle. Processes that are less rigorous than sterilization may include, for example, disinfection, sanitization, decontamination, cleaning, and the like. The sterilization processes provided for herein may be conducted for an effective period of time to achieve at least a 4 log reduction, or at least a 5 log reduction, or at least a 6 log reduction in the number of spores capable of reproduction, metabolism and/or growth. When at least a 6 log reduction is achieved, the process may be referred to as a sterilization process. When a 4 log reduction or a 5 log reduction is achieved, the process may be considered to be less rigorous than a sterilization, but nevertheless useful for various disinfection, sanitization, decontamination and/or cleaning applications. For purposes of this disclosure and the appended claims, the term “sterilization” is used to refer to a process that provides at least a 4 log reduction, at least a 5 log reduction, or at least a 6 log reduction in the number of spores capable of reproduction, metabolism and/or growth.

The inventive biological indicator may be formed by the deposition of an inoculum comprising the spores on the carrier. The inoculum may be dried on the carrier to form a spore deposit. Upon deposition on the carrier, the deposited inoculum may have a perimeter, and prior to being dried, the inoculum may be spread to expand the perimeter of the spore deposit. The inoculum deposited on the carrier may have a volume in the range from about 10 to about 1,000 microliters, or from about 20 to about 500 microliters, or from about 10 to about 50 microliters, or from about 10 to about 20 microliters. The inoculum may comprise water and the spores dispersed in the water. The inoculum may optionally further comprise a surfactant.

The water may comprise tap water, deionized water, distilled water, water purified by osmosis, or a mixture of two or more thereof.

The spores may comprise bacterial spores. The spores may comprise spores of the Bacillus or Clostridia genera. The spores may comprise spores of Geobacillus stearothermophilus, Bacillus atrophaeus, Bacillus sphaericus, Bacillus anthracis, Bacillus pumilus, Bacillus coagulans, Clostridium sporogenes, Clostridium difficile, Clostridium botulinum, Bacillus subtilis globigii, Bacillus cereus, Bacillus circulans, or a mixture of two or more thereof. The spores may comprise spores of Geobacillus stearothermophilus. The concentration of spores in the inoculum may range from about 1×10³ to about 1×10⁶ colony-forming units (cfu) per microliter (μl), or from about 1×10⁴ to about 1×10⁵ cfu/μl.

The surfactant may comprise any compound that lowers surface tension or provides greater wettability. The surfactant may comprise one or more wetting agents, emulsifiers, and/or dispersants. The surfactant may comprise one or more organic compounds that contain both hydrophobic groups and hydrophilic groups. The surfactant may comprise both a water insoluble component and a water soluble component. The surfactant may comprise one or more anionic, cationic, zwitterionic and/or nonionic compounds. The surfactant may comprise one or more alkanolamines, alkylarylsulfonates, amine oxides, poly(oxyalkylene)s, block copolymers comprising alkylene oxide repeat units, carboxylated alcohol ethoxylates, ethoxylated alcohols, alkyl phenols, ethoxylated alkyl phenols, ethoxylated amines, ethoxylated amides, oxiranes, ethoxylated fatty acids, ethoxylated fatty esters, ethoxylated oils, fatty esters, fatty acid amides, glycerol esters, glycol esters, sorbitan, sorbitan esters, imidazolines, lecithin, lignin, glycerides (e.g., mono-, di- and/or triglyceride), olefin sulfonates, phosphate esters, ethoxylated and/or propoxylated fatty acids and/or alcohols, sucrose esters, sulfates and/or alcohols and/or ethoxylated alcohols of fatty esters, sulfonates of dodecyl and/or tridecyl benzenes, sulfosuccinates, dodecyl and/or tridecyl benzene sulfonic acids, mixtures of two or more thereof, and the like. The surfactant may comprise ethanolamine, triethanolamine, octyldimethylamine oxide, nonylphenoxy poly(ethyleneoxy)ethanol, polyalkylene glycol, or a mixture of two or more thereof. The surfactant may comprise Tween 20, Tween 80 or Triton X-100. Tween 20 and Tween 80 are available from Sigma-Aldrich. Tween 20 is identified as a polyethylene glycol sorbitan monolaurate (CAS No. 9005-64-5). Tween 80 is identified as polyethylene glycol sorbitan monooleate (CAS No. 9005-65-6). Triton X-100 is a product of Dow identified as a polyethylene glycol (CAS No. 9002-93-1). The concentration of the surfactant in the inoculum may be in the range up to about 1% by weight, or from about 0.01 to about 1% by weight, or from about 0.01 to about 0.5% by weight, or from about 0.05 to about 0.15% by weigh, or from about 0.09 to about 0.11% by weight, or about 0.1% by weight.

The carrier may comprise a hydrophobic substrate. The carrier may comprise a non-porous material. The carrier may comprise a solid material. The carrier may comprise any material that does not dissolve or deteriorate during the sterilization or incubation processes. The carrier may comprise any material that does not combust with a high concentration of the oxidizing sterilant. The carrier may comprise metal, glass, ceramics, plastic, or a combination of two or more thereof. The metal may comprise aluminum or steel. The plastic may comprise a polyolefin, polystyrene, polycarbonate, polymethacrylate, polyacrylamide, polyimide, polyester, and the like. The carrier may comprise a film. The carrier may comprise a surface within a biological indicator, for example, a self-contained biological indicator (SCBI).

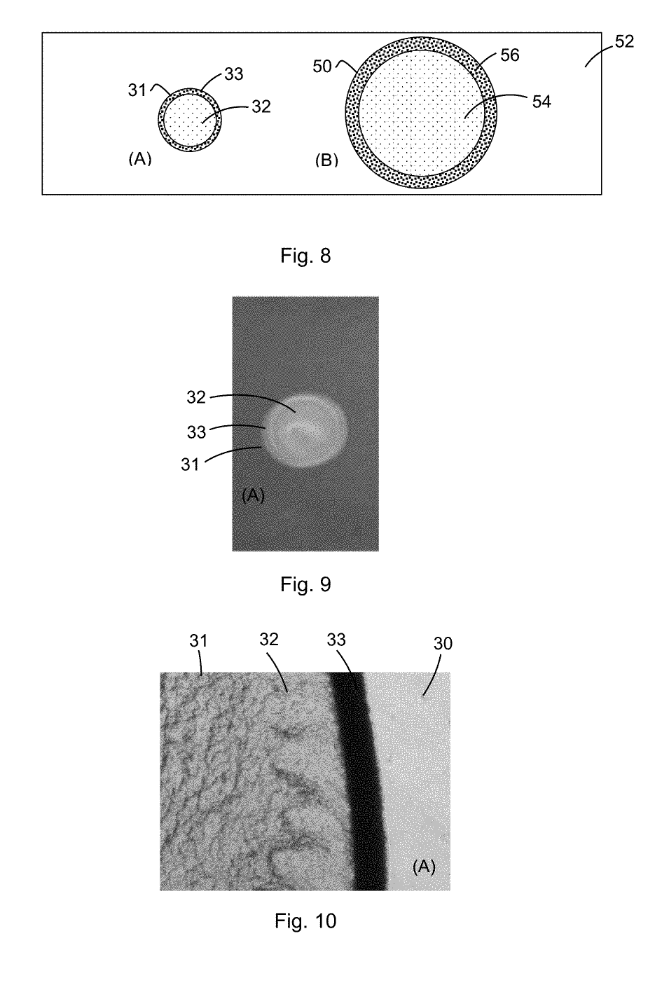

The spore deposit may have a central region and a peripheral region. See, for example, FIG. 8 wherein spore deposit 50 includes central region 54 and peripheral region 56. The spore deposit may separate into a central region and a peripheral region due to the fact that as the drop of inoculum dries, the spores tend to not distribute on the carrier in a uniform, monodispersed manner. Instead, as the drop dries, the spores tend to accumulate in the peripheral region of the spore deposit. As such, the concentration of spores in the peripheral region may be higher than in the central region. The overall total number of spores in the spore deposit may be in the range from about 1×10⁵ to about 5×10⁷ cfu, or from about 1×10⁶ to about 5×10⁶ cfu. The central region of the spore deposit may comprise a single spore layer with little spore stacking (for example, less than about 1% of the area of the central region comprising spores in a stacked configuration, or less than about 0.1% spore stacking) or no spore stacking. The peripheral region may contain spores wherein the spores reside in a stacked configuration. The central region may comprise at least about 95%, or at least about 96%, or at least about 97%, or at least about 98%, or at least about 99%, or at least about 99.5%, or at least about 99.8%, or at least about 99.9%, or at least about 99.95%, or at least about 99.99%, of the area of the spore deposit where the spores reside in a single spore layer. In an embodiment, less than about 5%, or less than about 4%, or less than about 3%, or less than about 2%, or less than about 1%, or less than about 0.5%, or less than about 0.1%, or less than about 0.05%, or less than about 0.01% of the area of the spore deposit comprises spores residing in a stacked configuration.

The carrier may be positioned in a biological indicator, for example, a self-contained biological indicator (SCBI). The carrier may comprise a surface in a compartment of a biological indicator. The carrier or interior surface of the biological indicator may be inoculated with the inoculum. The biological indicator may comprise a capped container with two separate compartments. One of the compartments may contain the inoculated carrier or an inoculated interior surface. The other compartment may contain a growth media. This compartment may comprise a frangible glass vial of growth media that can be broken just prior to incubation. In use, the biological indicator and the article to be sterilized are exposed to a sterilant during a sterilization process. Then following sterilization, the biological indicator is activated to allow for the test organism spores to come into contact with the growth media. The biological indicator may then be incubated to determine whether the sterilization process is effective.

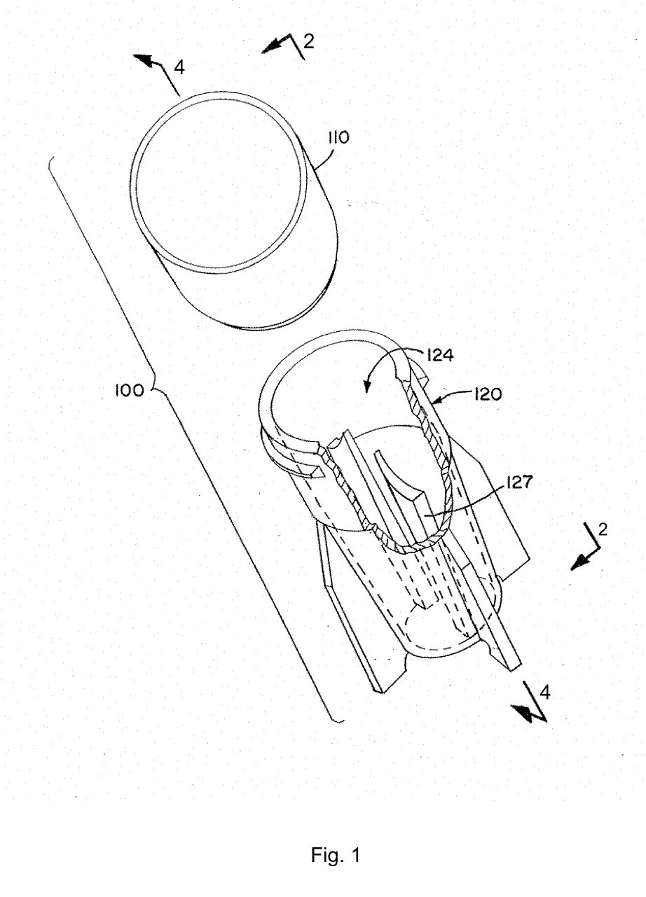

The biological indicator may comprise a SCBI, which may be in the form illustrated in FIGS. 1-4. Referring to FIGS. 1-4, SCBI 100 includes cap 110 which is configured for housing fluid 140. The fluid 140 contains a growth media. Cap 110, which is mounted on container 120, includes inner chamber 116. The inner chamber 116 has an opening 115 with a breakable barrier 130 overlying the opening 115. The fluid 140 is encapsulated within the inner chamber 116. The container 120 has an interior region 124 where carrier 190 is positioned. The carrier 190 may be inoculated as described above to form a spore deposit on the carrier 190.

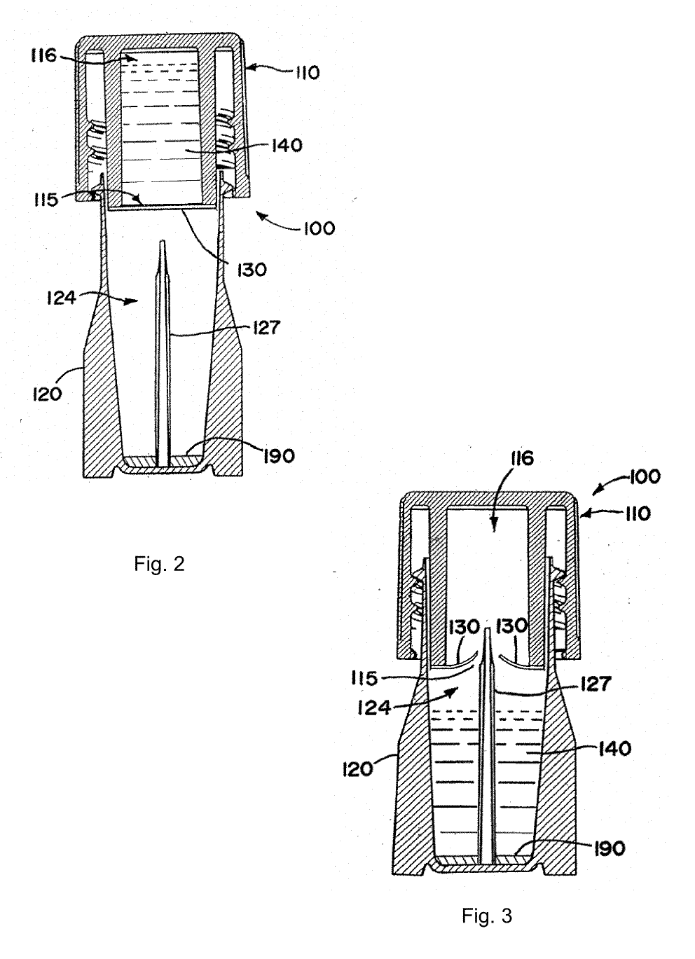

When used in a sterilization process, the cap 110 is held in an open position as illustrated in FIG. 2. The SCBI 100 and items to be sterilized are then subjected to the sterilization process. During the sterilization process, the sterilant passes through openings between the cap 110 and the container 120 and flows into the interior region 124 where it contacts and acts upon the test organism spores deposited on the carrier 190.

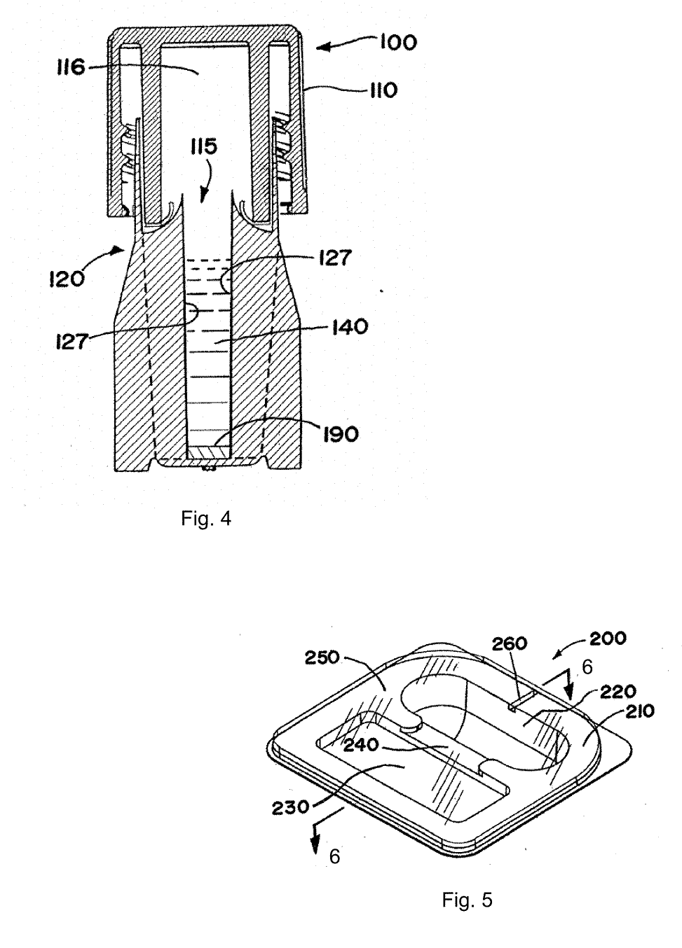

After the sterilization process is complete, the SCBI 100 is activated by screwing the cap 110 downward into a closed position as shown in FIGS. 3 and 4. This results in the breakable barrier 115 being broken by puncture member 127 to form broken barrier 130. (Note that two puncture members may be used in the embodiment shown in FIG. 4). Fluid 140, which contains a growth media, then flows into the container 120 in contact with the test organisms deposited on the carrier 190.

While in container 120, the test organisms spores and growth media may be incubated for a sufficient period of time to determine the viability of the spores. At the end of the incubation period, the SCBI may be evaluated to determine whether any spores survive the sterilization process. If the spores survive the sterilization process, the sterilization process is not considered to have been successful. On the other hand, if the spores are killed, then the sterilization process is considered to be successful.

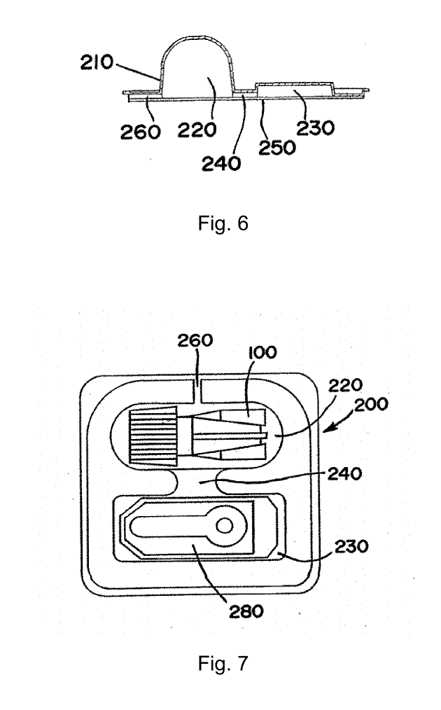

The SCBI 100 may be used with a test pack as depicted in FIGS. 5-7. Referring to FIGS. 5-7, test pack 200 includes base 210 containing recessed compartments 220 and 230. The recessed compartments 220 and 230 are in fluid communication with each other via internal channel 240. Cover 250 is attached to the base 210 and forms a sealed enclosure for the recessed compartments 220 and 230. External channel 260 provides a fluid communication between the sealed enclosure and an external environment. The SCBI 100 is positioned in recessed compartment 220. A chemical integrator and/or a chemical indicator 280 is positioned in recessed compartment 230. The external channel 260 is configured to allow a restricted flow of sterilant into the recessed compartments 220 and 230. The base 210 and cover 250 are otherwise impenetrable by the sterilant. The chemical integrator and/or chemical indicator 280 may undergo a change in color after it has been exposed to a sufficient quantity of sterilant in the sterilization medium for a sufficient period of time to indicate that the sterilization process has been completed. The sterilant also flows into the SCBI 100 in recessed compartment 220, and contacts the test organism spores in the SCBI 100.

Another modification of the biological indicator may be a rapid read or fast acting biological indicator. In this form the biological indicator may be mated to a dedicated instrument (reader) that detects early signals of test organism spore viability.

The biological indicator may be used to release loads or validate sterilization chamber functionality in healthcare settings. The biological indicator may also be used to determine if biological indicator waste has been properly decontaminated. In the scientific setting, the biological indicator may be used to validate the functionality of sterilization chambers, release loads of goods, or validate that a process meets required functionality. A valid biological indicator for a given process requires a specific resistance and therefore biological indicator manufacturers may strive to manufacture the biological indicator with targeted resistance characteristics.

The resistance for the biological indicator may be expressed as its D-value, which quantifies the time required to achieve inactivation of 90% of the population of test microorganisms, i.e., spores. The D-value may be measured in seconds. The D-value for the inventive biological indicator may be in the range from about 10 to about 50 seconds, or from about 15 to about 40 seconds, or from about 20 to about 35 seconds, or from about 25 to about 35 seconds. In comparison, commercially available biological indicators typically exhibit D-values in the range of about 2 to about 6 seconds. With the increased D-values provided by this invention, it is possible to subject the biological indicator to longer exposure times to a sterilant before an all kill of the spores on the biological indicator is achieved. This allows for extending the coverage of the sterilization cycle being tested using the inventive biological indicator.

The biological indicator may be used by subjecting it to the same sterilization medium and treatment as the articles for which sterile conditions may be sought. Sterilant (e.g., vaporous hydrogen peroxide, ethylene oxide, steam, etc.) may pass into the area where the biological indicator is located thereby exposing the biological indicator to the same sterilization process as the articles being sterilized. Following sterilization, growth media may be brought into contact with the biological indicator. The growth media may be in the form of a liquid. The growth media may comprise a buffered aqueous solution. Any procedure whereby the biological indicator is brought into contact with the growth media under conditions which allow for growth of test organisms, if any exists, may be used. The growth media may be present in the sterilization chamber in powder or tablet form and, after sterilization, sterile water may be added such that the biological indicator comes into contact with the aqueous incubation medium.

The growth media may comprise one or more nutrient sources. The nutrient source may be used to provide energy for the growth of any of the test organisms that may survive the sterilization process. Examples of the nutrient sources may include pancreatic digest of casein, enzymatic digest of soybean meal, sucrose, dextrose, yeast extract, L-cystine, and mixtures of two or more thereof.

A microbial growth indicator, which changes color or native state, in the presence of viable test organisms may be used with the growth media. The growth indicator may be dispersed or solubilized in the growth media and impart an initial color to the growth media. The growth indicator may also impart a color change in the growth media upon test organism growth. Growth indicators which may be employed include pH-sensitive dye indicators (such as bromothymol blue, bromocresol purple, phenol red, etc. or combinations thereof), oxidation-reduction dye indicators (such as methylene blue, etc.). The use of these microbial growth indicators may result in a change in color in response to a phenomenon of microorganism growth, such as changes in pH, oxidation-reduction potentials, enzymatic activity, as well as other indications of growth.

The growth media may further comprise one or more pH buffers, one or more neutralizers, one or more agents for maintaining osmotic equilibrium, or a mixture of two or more thereof.

The pH buffers may include K₂HPO₄, KH₂PO₄, (NH₄)₂HPO₄, 2,2-Bis(hydroxylmethyl)-2,2′,2″-nitrilothiethanol (Bis Tris), 1,3-Bis[tris(hydroxymethyl)methylamino] propane (Bis-Tris Propane), 4-(2-Hydroxyethyl)piperazine-ethanesulfonic acid (HEPES), 2-Amino-2-(hydroxymethyl)-1,3-propanediol (Trizma, Tris base), N-[Tris(hydroxymethyl)methyl]glycine (Tricine), Diglycine (Gly-Gly), N,N-Bis(2-hydroxyethyl)glycine (Bicine), N-(2-Acetamido)iminodiacetic acid (ADA), N-(2-Acetamido)-2-aminoethanesulfonic acid (aces), 1,4-Piperazinediethanesulfonic acid (PIPES), Beta-Hydroxy-4-morpholinepropanesulfonic acid (MOPSO), N,N-Bis(2-hydroxyethyl)-2-am inoethanesulfonic acid (BES), 3-(N-Morpholino)propanesulfonic acid (MOPS), 2-[(2-Hydroxy-1,1-bis(hydroxylmethyl)ethyl)amino]ethanesulfonic acid (TES), 3-(N,N-Bis[2-hydroxyethyl]amino)-2-hydroxypropanesulfonic acid (DIPSO), 4-(N-Morpholino)butanesulfonic acid (MOBS), 2-Hydroxy-3-[tris(hydroxymethyl)methylamino]-1-propanesulfonic acid (TAPSO), 4-(2-Hydroxyethyl)piperazine-1-(2-hydroxypropanesulfonic acid hydrate (HEPPSO), Piperazine-1,4-bis(2-hydroxypropanesulfonic acid) dihydrate (POPSO), 4-(2-Hydroxyethyl)-1-piperazine propanesulfonic acid (EPPS), N-(2-Hydroxyethyl)piperazine-N′-(4-butanesulfonic acid) (HEPBS), [(2-Hydroxy-1,1-bis(hydroxymethyl)ethyl)amino]-1-propanesulfonic acid (TAPS), 2-Amino-2-methyl-1,3-propanediol (AMPD), N-tris(Hydroxymethyl)methyl-4-am inobutanesulfonic acid (TABS), N-(1,1-Dimethyl-2-hydroxyethyl)-3-amino-2-hydroxypropanesulfonic acid (AMPSO), 2-(Cyclohexylamino)ethanesulfonic acid (CHES), 3-(Cyclohexylamino)-2-hydroxyl-1-propanesulfonic acid (CAPSO), 2-Amino-2-methyl-1-propanol (AMP), 3-(Cyclohexylamino)-1-propanesulfonic acid (CAPS), 4-(Cyclohexylamino)-1-butanesulfonic acid (CABS), 2-(N-Morpholino)ethanesulfonic acid hydrate (MES), N-(2-Acetamido)-2-aminoethanesulfonic acid (ACES), and mixtures of two or more thereof.

The neutralizers may include but are not limited to sodium thioglycollate, sodium thiosulfate, catalase, sodium bisulfate, sodium bisulfite lecithin, polysorbate 20, polysorbate 80, calcium bicarbonate, and mixtures of two or more thereof.

The agents for maintaining osmotic equilibrium may include sodium salt, potassium salts, magnesium salts, manganese salts, calcium salts, metallic salts, sodium chloride, potassium chloride, magnesium sulfate, iron chloride, and mixtures of two or more thereof.

The growth media may comprise an aqueous composition comprising: water; from about 0.01 to about 100 grams per liter (g/I), or from about 0.1 to about 50 g/l, of one or more nutrient sources; from about 1.0×10-5 to about 10 g/l, or from about 1.0×10-4 to about 1.0 g/l of one or more microbial growth indicators; up to about 5000 g/l, or from about 0.001 to about 5000 g/l, or from about 0.1 to about 1000 g/l, of one or more pH buffers; up to about 100 g/l, or from about 0.01 to about 100 g/l, or from about 0.1 to about 50 g/l, of one or more neutralizers; up to about 50 g/l, or from about 0.1 to about 50 g/l, or from about 0.1 to about 25 g/l, of one or more agents for maintaining osmotic equilibrium.

### Example 1

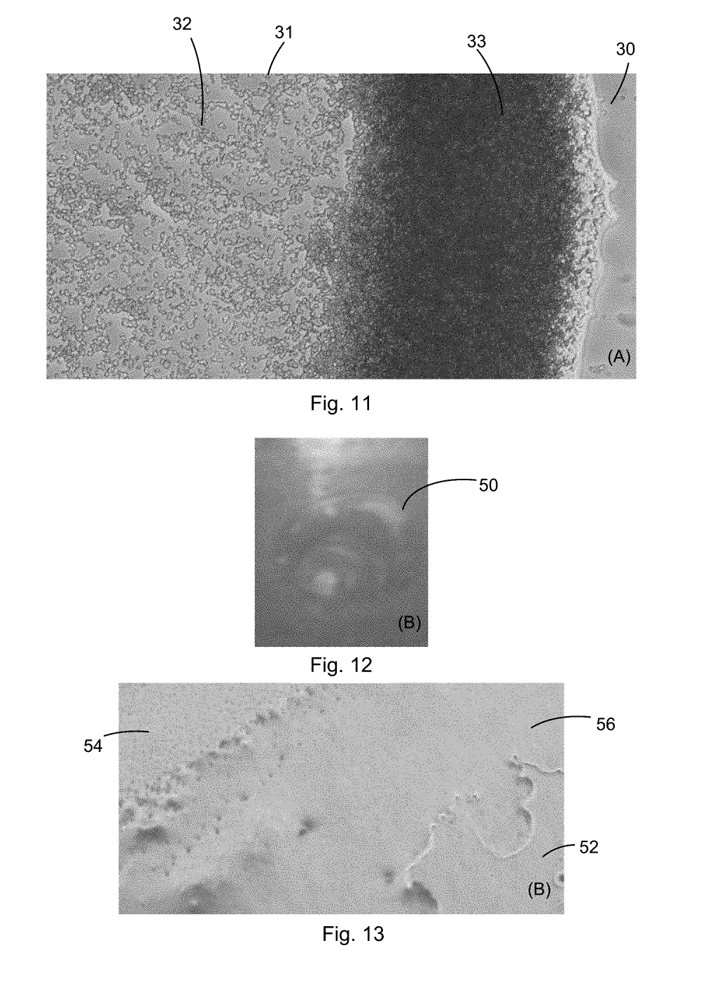

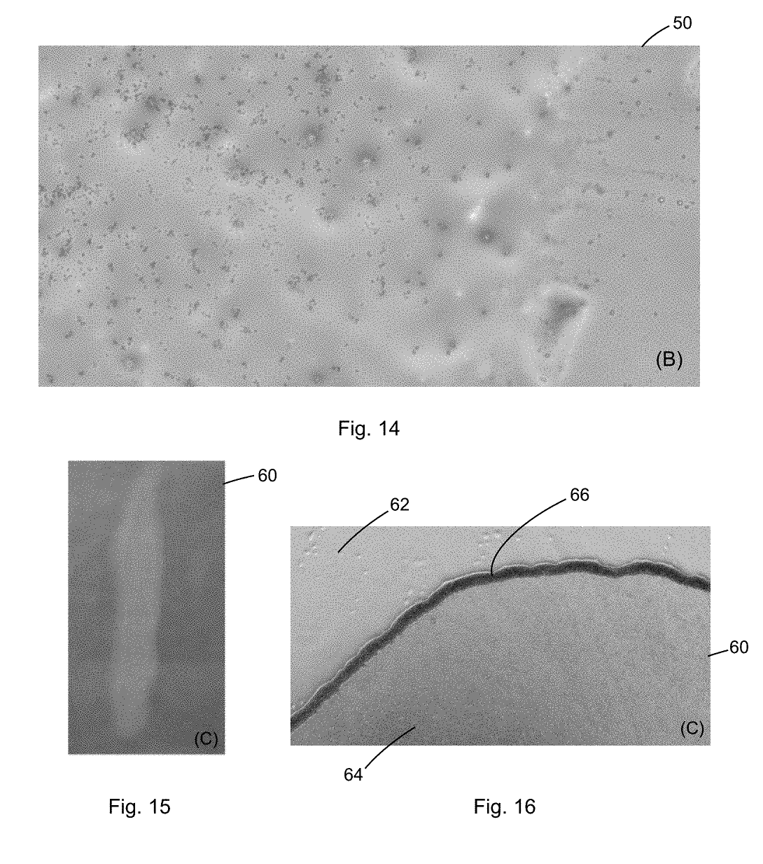

Two 20-microliter drops are formed using inoculums containing Geobacillus stearothermophilus spores. The first of these is drop (A) which contains the spores dispersed in water. The second is drop (B) which is the same as drop (A) except that it contains a surfactant. The surfactant is Triton X-100. The concentration of spores in each inoculum is 1×10⁵ cfu per microliter. The concentration of surfactant in (B) is 0.1% by weight. The drops (A) and (B) are dried to form spore deposits. The spore deposit from droplet (A) is illustrated or shown in FIGS. 8-11. Referring to FIGS. 8-11, spore deposit 31 is formed on glass plate 52, and includes central region 32 and peripheral region 33. The spore deposit from drop (B) is shown in FIGS. 8 and 12-14. Referring to FIGS. 8 and 12-14, spore deposit 50 is formed on glass plate 52 and includes central region 54 and peripheral region 56. As shown in Table 1, use of an inoculum (B) containing the surfactant results in a 90% increase in the circumference of the spore deposit as compared to the inoculum (A) without surfactant. This is indicative of less spore stacking for spore deposits from the inoculum (B) using a surfactant as compared to spore deposits from the inoculum (A) without surfactant. The surfactant reduces surface tension which spreads out the inoculum and results in a peripheral region 56 showing little or no spore stacking. For the spore deposit 50, 99-100% of the area of the spore deposit comprises spores that reside in a single spore layer.

TABLE 1
Circumference of spore deposit from a 20 microliter drop of inoculum
(B) containing a surfactant or inoculum (A) without a surfactant

| (A) Without | (B) With | Increase in |
|---|---|---|
| surfactant | surfactant | circumference |
| 1.333 cm | 2.53 cm | 90%* |

*(2.53 − 1.333 = 1.197)/1.333 × 100 = 90%

The foregoing shows that the use of a surfactant results in an increase in the circumference of the spore deposit. The increase in the spreading of the spore deposit with the surfactant eliminates (or nearly eliminates) the presences of stacked spores in the spore deposit.

### Example 2

Spores of Geobacillus stearothermophilus are suspended in water to form an inoculum. The concentration of spores in the inoculum is 1×10⁵ cfu per microliter. A 10 microliter drop (C) is deposited on a glass slide. The drop (C) is spread out manually to further extend the perimeter and dried to form a spore deposit with an increased circumference. The resulting spore deposit is shown in FIGS. 15 and 16 as spore deposit 60. Referring to FIGS. 15 and 16, spore deposit 60 is formed on glass plate 62. Spore deposit 60 includes central region 64 and peripheral region 66. For the spore deposit 60, 95-99% of the area of the spore deposit comprises spores that reside in a single spore layer, the single spore layer being in the central region 64.

### Example 3

D-value is the time under exposure to a sterilant required to reduce a viable spore population by 90%. This value can be measured in seconds. The biological indicators provided herein exhibit desired increased D-values when exposed to vaporous hydrogen peroxide (VHP) sterilization cycles. These biological indicators require a longer exposure before all kill is achieved thus extending coverage for the biological indicator into the sterilization cycle being tested. This is shown in Table 2 wherein spore deposits are tested using inoculums containing Geobacillus stearothermophilus spores at a concentration of 2×10⁶ cfu per deposit. A single spore deposit (D) is formed using a 20 microliter drop containing 0.1% by weight Tween 80. 95-98% of the area of deposit (D) has spores residing in a single spore layer. A single spore deposit (E) is formed using a 20 microliter drop containing 0.1% by weight Tween 20. 95-98% of the area of deposit (E) has spores residing in a single spore layer. A single spore deposit (F) is formed using a 20 microliter drop containing 0.1% by weight Triton X-100. 99-100% of the area of deposit (F) has spores residing in a single spore layer. The D-values shown in Table 2 exhibit an improvement over prior art biological indicators where the D-values are typically about 2-6 seconds.

TABLE 2
D-Values for spore deposits (D), (E), and (F)

| (D) One 20 | (E) One 20 | (F) One 20 |
|---|---|---|
| microliter | microliter | microliter |
| drop with | drop with | drop with |
| 0.1% Tween 80 | 0.1% Tween 20 | 0.1% Triton X100 |
| 27.3 seconds | 28.0 seconds | 33.0 seconds |

While the invention has been explained in relation to various embodiments, it is to be understood that various modifications thereof will become apparent to those skilled in the art upon reading the specification. Therefore, it is to be understood that the invention disclosed herein includes any such modifications that may fall within the scope of the appended claims.

1. A biological indicator, comprising:
a carrier; and
a spore deposit on the carrier, wherein at least about 95% of the area of the spore deposit comprises spores residing in a single spore layer.

2. The biological indicator of claim 1 wherein the carrier comprises a hydrophobic substrate.

3. The biological indicator of claim 1 wherein the carrier comprises metal, glass, ceramics, plastic, or a combination of two or more thereof.

4. The biological indicator of claim 1 wherein the spore deposit is derived from an inoculum comprising water, spores and a surfactant.

5. The biological indicator of claim 4 wherein the water comprises tap water, deionized water, distilled water, water purified by osmosis, or a mixture of two or more thereof.

6. The biological indicator of claim 4 wherein the surfactant comprises an organic compound that contains hydrophobic groups and hydrophilic groups.

7. The biological indicator of claim 4 wherein the surfactant comprises an anionic, cationic, zwitterionic and/or nonionic compound.

8. The biological indicator of claim 4 wherein the surfactant comprises: an alkanolamine; alkylarylsulfonate; amine oxide; poly(oxyalkylene); block copolymer comprising alkylene oxide repeat units; carboxylated alcohol ethoxylate; ethoxylated alcohol; alkyl phenol; ethoxylated alkyl phenol; ethoxylated amine; ethoxylated amide; oxirane; ethoxylated fatty acid; ethoxylated fatty ester; ethoxylated oil; fatty ester; fatty acid amide; glycerol ester; glycol ester; sorbitan; sorbitan ester; imidazoline; lecithin; lignin; glyceride; olefin sulfonate; phosphate ester; ethoxylated fatty acid; propoxylated fatty acid; ethoxylated fatty alcohol; propoxylated fatty alcohol; sucrose ester; sulfate, alcohol and/or ethoxylated alcohol of a fatty ester; sulfonate of dodecyl and/or tridecyl benzene; sulfosuccinate; doecyl and/or tridecyl benzene sulfonic acid; or a mixture of two or more thereof.

9. The biological indicator of claim 4 wherein the surfactant comprises ethanolamine, triethanolamine, octyldimethylamine oxide, nonylphenoxy poly(ethyleneoxy)ethanol, polyalkylene glycol, or a mixture of two or more thereof.

10. The biological indicator of claim 4 wherein the concentration of the surfactant in the inoculum is in the range from about 0.001 to about 1% by weight.

11. The biological indicator of claim 1 wherein the biological indicator is in a first compartment of an indicator device, the first compartment being adapted to permit the spores to be brought into contact with a sterilant during a sterilization process; and the indicator device further comprises a second compartment containing a growth media, the second compartment being adapted to maintain the growth media separate from the spores during the sterilization process, and to permit the growth media to contact the spores after the sterilization process is completed.

12. The biological indicator of claim 11 wherein the indicator device is positioned in a test pack.

13. The biological indicator of claim 1 wherein the spores comprise bacterial spores.

14. The biological indicator of claim 1 wherein the spores comprise spores of the Bacillus or Clostridia genera.

15. The biological indicator of claim 1 wherein the spores comprise spores of Geobacillus stearothermophilus, Bacillus atrophaeus, Bacillus sphaericus, Bacillus anthracis, Bacillus pumilus, Bacillus coagulans, Clostridium sporogenes, Clostridium difficile, Clostridium botulinum, Bacillus subtilis globigii, Bacillus cereus, Bacillus circulans, or a mixture of two or more thereof.

16. The biological indicator of claim 1 wherein the spores comprise Geobacillus stearothermophilus spores.

17. The biological indicator of claim 1 wherein the spore deposit has a total number of spores in the range from about 1×10⁶ to about 1×10⁸ colony forming units.

18. The biological indicator of claim 4 wherein the concentration of spores in the inoculum is in the range from about 1×10³ to about 1×10⁶ colony forming units per microliter.

19. The biological indicator of claim 4 wherein the spore deposit is formed by the deposition of an inoculum on the carrier, the inoculum having a volume in the range from about 10 to about 1000 microliters.

20. The biological indicator of claim 1 wherein the biological indicator has a D-value in the range from about 10 to about 50 seconds.

21. A sterilization process, comprising: exposing an article to be sterilized and the biological indicator of claim 1 to a sterilant.

22. The process of claim 21 wherein the sterilant comprises vaporous hydrogen peroxide, ethylene oxide, steam, peracetic acid, ozone, or a combination of two or more thereof.

23. A process for determining the effectiveness of a sterilization process, comprising:
exposing an article to be sterilized and the biological indicator of claim 1 to a sterilant; and
incubating the biological indicator in the presence of a growth media to determine whether the sterilization process is effective.

24. A process for making a biological indicator, comprising:
(A) forming an inoculum comprising spores dispersed in water;
(B) depositing the inoculum on a carrier; and
(C) drying the inoculum to form a spore deposit on the carrier, wherein at least about 95% of the spore deposit comprises spores residing in a single spore layer.

25. The process of claim 24 wherein the spore deposit has a total number of spores in the range from about 1×10⁶ to about 1×10⁸ colony forming units.

26. The process of claim 24 wherein the carrier comprises a hydrophobic substrate.

27. The process of claim 24 wherein the carrier comprises metal, glass, ceramics, plastic, or a combination of two or more thereof.

28. The process of claim 24 wherein the water comprises tap water, deionized water, distilled water, water purified by osmosis, or a mixture of two or more thereof.

29. The process of claim 24 wherein the inoculum deposited on the carrier forms a perimeter on the carrier, and prior to being dried the inoculum is spread to extend the perimeter.

30. The process of claim 24 wherein the inoculum further comprises a surfactant.

31. The process of claim 30 wherein the surfactant comprises an organic compound that contains hydrophobic groups and hydrophilic groups.

32. The process of claim 30 wherein the surfactant comprises an anionic, cationic, zwitterionic and/or nonionic compound.

33. The process of claim 30 wherein the surfactant comprises: an alkanolamine; alkylarylsulfonate; amine oxide; poly(oxyalkylene); block copolymer comprising alkylene oxide repeat units; carboxylated alcohol ethoxylate; ethoxylated alcohol; alkyl phenol; ethoxylated alkyl phenol; ethoxylated amine; ethoxylated amide; oxirane; ethoxylated fatty acid; ethoxylated fatty ester; ethoxylated oil; fatty ester; fatty acid amide; glycerol ester; glycol ester; sorbitan; sorbitan ester; imidazoline; lecithin; lignin; glyceride; olefin sulfonate; phosphate ester; ethoxylated fatty acid; propoxylated fatty acid; ethoxylated fatty alcohol; propoxylated fatty alcohol; sucrose ester; sulfate, alcohol and/or ethoxylated alcohol of a fatty ester; sulfonate of dodecyl and/or tridecyl benzene; sulfosuccinate; doecyl and/or tridecyl benzene sulfonic acid; or a mixture of two or more thereof.

34. The process of claim 30 wherein the surfactant comprises ethanolamine, triethanolamine, octyldimethylamine oxide, nonylphenoxy poly(ethyleneoxy)ethanol, polyalkylene glycol, or a mixture of two or more thereof.

35. The process of claim 30 wherein the concentration of the surfactant in the inoculum is in the range from about 0.001 to about 1% by weight.

36. The process of claim 24 wherein the spores comprise bacterial spores.

37. The process of claim 24 wherein the spores comprise spores of the Bacillus or Clostridia genera.

38. The process of claim 24 wherein the spores comprise spores of Geobacillus stearothermophilus, Bacillus atrophaeus, Bacillus sphaericus, Bacillus anthracis, Bacillus pumilus, Bacillus coagulans, Clostridium sporogenes, Clostridium difficile, Clostridium botulinum, Bacillus subtilis globigii, Bacillus cereus, Bacillus circulans, or a mixture of two or more thereof.

39. The process of claim 24 wherein the spores comprise Geobacillus stearothermophilus spores.

40. The process of claim 24 wherein the inoculum has a concentration of spores in the range from about 1×10³ to about 1×10⁶ colony forming units per microliter.

41. The process of claim 24 wherein the inoculum has a volume in the range from about 10 to about 1000 microliters.
